(19) United States
(12) Patent Application Publication
(10) Pub. No.: US 2024/0210225 A1
(43) Pub. Date: Jun. 27, 2024

(54) THERMAL FLOW METER WITH AUTOMATIC GAS DETECTION

(71) Applicants: TrueDyne Sensors AG, Reinach (CH); Innovative Sensor Technology IST AG, Ebnat-Kappel (CH)

(72) Inventors: Christof Huber, Bern (CH); Sandro Schwab, Oftringen (CH); Ralf Emanuel Bernhardsgrütter, Sulgen (CH); Fabio Schraner, Basel (CH); Josua Ritter, Reinach (CH); Jakob Schaab, Zürich (CH); Marcel Giger, Wattwil (CH); Jürgen Dorsch, Ebnat-Kappel (CH)

(21) Appl. No.: 18/390,776

(22) Filed: Dec. 20, 2023

(30) Foreign Application Priority Data
Dec. 23, 2022 (DE) 20 2022 107 233.7

(51) Int. Cl. G01F 1/684; G01N 9/00; G01N 33/00

(52) U.S. Cl. CPC G01F 1/684; G01N 9/002; G01N 33/0027

(57) ABSTRACT

A thermal flow meter for measuring a flow rate of a gas includes: a measurement channel for guiding the gas between a channel inlet and a channel outlet; a thermal flow sensor disposed in the measurement channel; a density sensor including an oscillator operable to be acted upon by the gas; a temperature sensor element configured to determine a gas temperature; a pressure sensor element configured to determine a gas pressure; and a measuring-operating circuit configured to: determine a density value of the gas based on a natural frequency of the oscillator; identify a composition of the gas based on the density value, the gas pressure, and the gas temperature; and as a function of the composition, output a flow rate measured value corrected with respect to a cross-sensitivity of the flow rate to the composition of the gas based on flow rate-dependent signals of the thermal flow sensor.

### CROSS-REFERENCE TO RELATED APPLICATION

The present application is related to and claims the priority benefit of German Patent Application No. 20 2022 107 233.7, filed Dec. 23, 2022, the entire contents of which are incorporated herein by reference.

### TECHNICAL FIELD

The present disclosure relates to a thermal flow meter with automatic gas detection, in particular, for detecting the composition of gas mixtures, in particular binary gas mixtures, and/or pure gases.

### BACKGROUND

Thermal flow meters are usually adjusted to a particular medium or gas because the ascertained measurement value is influenced by the thermal conductivity and heat capacity of the gas. Thus, a specially calibrated sensor is required for each gas, or correction factors for various gases are specifically stored in the sensor by the manufacturer. Consequently, which gas is currently being measured must always first be communicated to the sensor. However, if the gas composition changes during a measurement, the current sensors are not able to ascertain the flow correctly.

### SUMMARY

The object of the present disclosure is, therefore, to find a remedy here. The object is achieved according to a flow meter according to the present disclosure.

A thermal flow meter according to the present disclosure for measuring a flow rate of a gas comprises: a measurement channel for guiding the gas between a channel inlet and a channel outlet; a thermal flow sensor which is arranged in the measurement channel; a density sensor which has an oscillator which can be acted upon by the gas, wherein a natural frequency of an oscillation mode of the oscillator depends upon the density of the gas; a temperature sensor element for ascertaining a gas temperature; and a pressure sensor element for ascertaining a gas pressure of the gas; and a measuring and operating circuit for operating the thermal flow sensor and the density sensor, wherein the measuring and operating circuit is configured to determine a density measurement value of the gas on the basis of the natural frequency of the oscillator and to identify the composition of the gas on the basis of the density measurement value, the gas pressure, and the gas temperature, and, as a function of the composition on the basis of flow-dependent signals of the thermal flow sensor, to output a flow measurement value corrected with respect to a cross-sensitivity to the composition of the gas.

In an embodiment according to the present disclosure, the measuring and operating circuit is configured to identify the composition of the gas, assuming a binary gas mixture of known components and/or a pure gas.

In an embodiment according to the present disclosure, the oscillator comprises a quartz tuning fork or a cantilever oscillator.

In an embodiment according to the present disclosure, the thermal flow sensor and/or the density sensor have a MEMS sensor element.

In an embodiment according to the present disclosure, the thermal flow sensor and the density sensor have a respective MEMS sensor element, wherein the two MEMS sensor elements are arranged on a common measuring board.

In an embodiment according to the present disclosure, the pressure sensor element and/or the temperature sensor element each comprise a MEMS sensor element.

In an embodiment according to the present disclosure, the measurement channel extends through a measurement chamber in which the density sensor, the temperature sensor element, and the pressure sensor element are arranged and can be acted upon by the gas.

In an embodiment according to the present disclosure, the thermal flow meter furthermore comprises a housing body through which the measurement channel extends, wherein the housing body has a mounting surface, wherein the mounting surface has openings which communicate with the measurement channel, wherein the sensor elements which can be acted upon by the gas are arranged, supported by the measuring board, in the openings.

In an embodiment according to the present disclosure, the measuring board seals the openings in a gas-tight manner.

In an embodiment according to the present disclosure, the thermal flow sensor comprises the temperature sensor element which is used to sense the gas temperature.

In an embodiment according to the present disclosure, the thermal flow meter furthermore comprises at least one further sensor element for sensing a measured variable of the gas, which is selected from a list comprising: moisture, viscosity, thermal conductivity, heat capacity. With each of the further sensor elements, a further property of the gas can be ascertained so that, with one further sensor, ternary gas mixtures and, with two further sensors, quaternary gas mixtures can thus also be characterized with regard to their composition in order to correct corresponding cross-sensitivities in the flow measurement.

### BRIEF DESCRIPTION OF THE DRAWINGS

The present disclosure will now be explained in more detail on the basis of the exemplary embodiments shown in the figures. In the figures:

FIG. 1 shows exemplary measurement data for flow measurement with gas detection;

FIG. 2a shows a schematic longitudinal section through an exemplary embodiment of a flow meter according to the present disclosure along the line A-A in FIG. 2b; and

FIG. 2b shows a schematic cross-sectional view through the exemplary embodiment of a flow meter according to the present disclosure along the line B-B in FIG. 2a.

### DETAILED DESCRIPTION

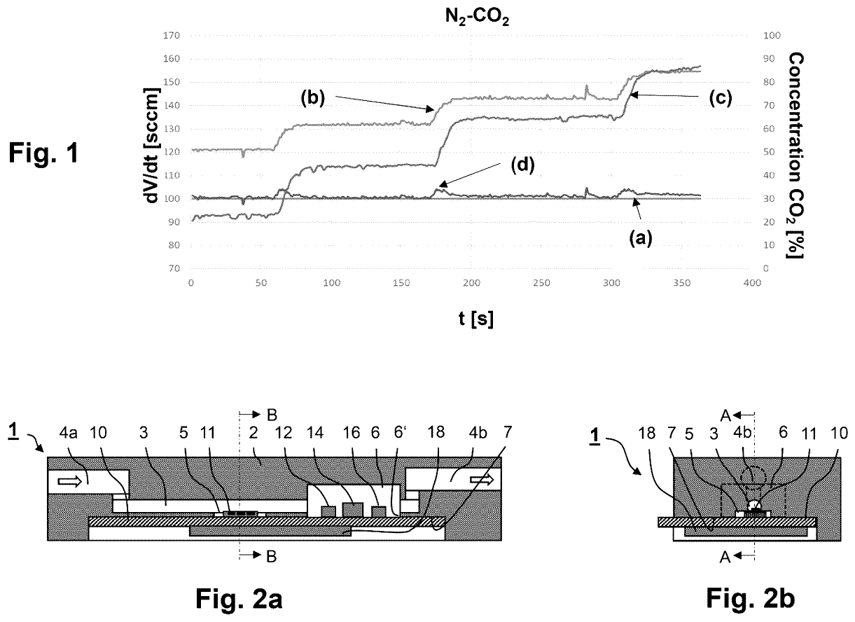

The operating principle of the flow meter according to the present disclosure is explained with reference to the graph in FIG. 1, which shows example measurement data for gas mixtures consisting of carbon dioxide and nitrogen in various compositions, including curves (a), (b), (c) and (d). The curve (a) shows the actual flow rate of the gas mixtures, which was at a constant 100 standard cubic centimeters per minute (sccm). In the course of the measurements for this diagram, the proportion of carbon dioxide was increased stepwise from 20% to over 80%. A conventional thermal flow sensor calibrated to the initial mixing ratio of 20% carbon dioxide and 80% nitrogen cannot handle this change in the gas composition and produces the measurement data shown in curve (b), which is shifted here by a constant offset of 20 sccm in order to rectify the diagram, i.e., the initial 120 sccm in curve (b) corresponds to 100 sccm measured, and the 155 sccm shown after 350 s correspond to 135 sccm measured. Insofar as a flow rate of 100 sccm was actually given here, a measurement error of 35% is present here due to the cross-sensitivity of the thermal flow sensor to the composition of the gas.

In order to be able to compensate for this cross-sensitivity, the flow meter according to the present disclosure includes a density sensor, the measurement data of which make it possible to ascertain the composition of a binary gas mixture of known components, wherein the result of this ascertainment is shown in curve (c). With knowledge of the actually current gas composition, the cross-sensitivity of the thermal flow sensor to the composition of the gas can be compensated for, which ultimately results in the measurement data shown in curve (d), which match the actual flow rate of 100 (sccm) very well. In the case of changes in the gas composition, a delay of a few seconds is given until the correct composition is detected. During this time, there are slight deviations of the measured flow rates from the actual flow rate, as can be seen in curve (d).

In comparison to the data according to the conventional thermal flow sensor in curve (b), the measurement data sensed by the flow meter according to the present disclosure demonstrate a considerable improvement. If various pure gases are present in a process plant, which are used alternately, they can likewise be identified by means of the density sensor in order to correct the flow rate with respect to the cross-sensitivity of the thermal flow measurement to the gas composition. The corresponding compensation functions with gas-dependent parameters are stored in a working memory of the measuring and operating circuit.

The exemplary embodiment shown in FIGS. 2a and 2b of a thermal flow meter 1 according to the present disclosure comprises a housing body 2 in which several cavities are provided. The latter comprise a measurement channel 3, which communicates with a channel inlet 4a, and a channel outlet 4b, via which the meter can be connected to a process line. A further cavity is a measurement chamber 6 through which the measurement channel 3 extends. The measurement chamber 6 has larger, clear cross-sections than the measurement channel 3 in order to provide space for sensor elements. The measurement chamber 6 has an opening 6′ towards a mounting surface 7 of the housing body 2. Likewise, the measurement channel 3 has an opening 5 to the mounting surface 7 at a distance from the measurement chamber 6. A measuring board 10, on which sensor elements are mounted and which communicate with the measurement channel 3 or the measurement chamber 6 through the openings 5, 6′, is in a sealed contact with the mounting surface 7.

A thermal flow sensor 11, which comprises a heating element and two temperature sensor elements, is positioned in the opening 5 to the measurement channel 3, wherein the heating element is arranged between the temperature sensor elements in the flow direction. The thermal flow sensor 11 is in particular designed as a micro-electromechanical systems (MEMS) sensor element. A density sensor 12, an absolute pressure sensor 14, and a temperature sensor 16 are furthermore arranged on the measuring board 10, wherein these three sensors are designed as MEMS sensor elements. The density sensor 12 comprises an oscillator, which may comprise a quartz tuning fork.

The flow meter 1 according to the present disclosure furthermore comprises a measuring and operating circuit 18 which, in particular, comprises a microcontroller, which is configured to operate the sensor elements and to evaluate the measurement signals thereof. The algorithms and parameters for identifying a gas composition on the basis of the signals of the density sensor 12 and of the pressure sensor 14 and the temperature sensor 16 are stored in a (program) memory of the microcontroller 18, as are the algorithms and gas-specific parameters for compensating for the cross-sensitivity of the thermal flow measurement with respect to the gas composition.

1. A thermal flow meter for measuring a flow rate of a gas, the thermal flow meter comprising:
a measurement channel configured to guide the gas between a channel inlet and a channel outlet;
a thermal flow sensor disposed in the measurement channel;
a density sensor including an oscillator operable to be acted upon by the gas, wherein a natural frequency of an oscillation mode of the oscillator depends upon the density of the gas;
a temperature sensor element configured to determine a gas temperature of the gas;
a pressure sensor element configured to determine a gas pressure of the gas; and
a measuring and operating circuit configured to:
operate the thermal flow sensor and the density sensor;
determine a density measurement value of the gas based on the natural frequency of the oscillator;
identify a composition of the gas based on the density measurement value, the gas pressure, and the gas temperature; and
as a function of the composition, output a flow rate measured value corrected with respect to a cross-sensitivity of the flow rate to the composition of the gas based on flow rate-dependent signals of the thermal flow sensor.

2. The thermal flow meter according to claim 1, wherein the measuring and operating circuit is further configured to identify the composition of the gas, assuming the gas consists of a binary gas mixture of known components and/or a pure gas.

3. The thermal flow meter according to claim 1, wherein the oscillator comprises a quartz tuning fork or a cantilever beam oscillator.

4. The thermal flow meter according to claim 1, wherein the thermal flow sensor and/or the density sensor comprise a micro-electromechanical systems (MEMS) sensor element.

5. The thermal flow meter according to claim 1, wherein the thermal flow sensor and the density sensor each comprise a MEMS sensor element, wherein the two MEMS sensor elements are arranged on a common measuring board.

6. The thermal flow meter according to claim 1, wherein the pressure sensor element and/or the temperature sensor element comprise a MEMS sensor element.

7. The thermal flow meter according to claim 1, wherein the measurement channel extends through a measurement chamber in which the density sensor, the temperature sensor element, and the pressure sensor element are arranged such that each of the density sensor, the temperature sensor element, and the pressure sensor element can be acted upon by the gas.

8. The thermal flow meter according to claim 7, where in each of the thermal flow sensor, the density sensor, the temperature sensor element, and the pressure sensor element comprise a MEMS sensor element.

9. The thermal flow meter according to claim 7, further comprising a housing body through which the measurement channel extends, wherein the housing body includes a mounting surface, wherein the mounting surface includes openings which communicate with the measurement channel, and wherein the thermal flow sensor, the density sensor, the temperature sensor element, and the pressure sensor element, which each can be acted upon by the gas, are arranged in the openings supported by the measuring board.

10. The thermal flow meter according to claim 9, wherein the measuring board is configured such that the measuring board seals the openings.

11. The thermal flow meter according to claim 1, wherein the thermal flow sensor comprises a temperature sensor element configured to determine a temperature measured value, whose measurement value is included as a gas temperature in calculating the density of the gas.

12. The thermal flow meter according to claim 1, further comprising at least one further sensor element configured to detect a measured variable of the gas, which measured variable is selected from: a moisture, a viscosity, a thermal conductivity, and a heat capacity.

13. The thermal flow meter according to claim 12, wherein the measuring and operating circuit is further configured to identify the composition of the gas, assuming the gas consists of a gas mixture of known components, wherein the number of known components is equal to the number of the at least one further sensor elements plus two.
